(19) United States
(12) Patent Application Publication
(10) Pub. No.: US 2025/0352684 A1
(43) Pub. Date: Nov. 20, 2025

(54) CLOUD-MANAGED AUTONOMOUS MEDICAL VEHICLE FOR DISINFECTION

(71) Applicant: FAR EASTONE TELECOMMUNICATIONS CO., LTD., Taipei City (TW)

(72) Inventors: CHIA-JUI YANG, Taipei City (TW); CHUN-CHIEH KUO, New Taipei City (TW); HUA-PEI CHIANG, Taipei City (TW); SHUI-SHU HSIAO, Taipei City (TW); CHYI-DAR JANG, Taipei City (TW); TSUNG-JEN WANG, Taipei City (TW); CHANG-HUNG HSU, Taipei City (TW); KUN-YAN LI, Taipei City (TW); CHI-EN CHIEN, New Taipei City (TW); HAO CHEN, New Taipei City (TW)

(21) Appl. No.: 19/069,946

(22) Filed: Mar. 4, 2025

(30) Foreign Application Priority Data
May 17, 2024 (TW) 113118477

(51) Int. Cl. A61L 2/18; A61L 2/24

(52) U.S. Cl. CPC A61L 2/18; A61L 2/24; A61L 2202/24

(57) ABSTRACT

A cloud-managed autonomous medical vehicle for disinfection includes a mobile carrier and also includes nozzles, a detector, and a nozzle controller, which are arranged on the mobile carrier. A disinfectant liquid container is disposed on the mobile carrier and filled with disinfectant liquid. The nozzle is used to spray the disinfectant liquid. The nozzles are respectively connected with motors, whereby the nozzle may be switched to different spraying modes. The detector scans ambient environment to detect the concentration of organic or inorganic materials and transmits the results to a cloud server. While the concentration is higher/lower than the preset value, the cloud server sends out a spray start/stop instruction. While receiving the spray start instruction, the nozzle controller switches the motor to a spraying mode for spraying operation. While receiving a spray stop instruction, the nozzle controller turns off the motor to stop spraying.

### BACKGROUND OF THE INVENTION

This application claims priority for the TW Application No. 11/311,8477 filed on 17 May 2024, the content of which is incorporated by reference in its entirely.

### FIELD OF THE INVENTION

The present invention relates to a disinfection apparatus, particularly to a cloud-managed autonomous medical vehicle for disinfection.

### DESCRIPTION OF THE PRIOR ART

Many respiratory diseases mainly spread through droplets or contagion, including tuberculosis, coronavirus pandemic (COVID 19), and severe acute respiratory syndrome (SARS). Wearing a medical face mask may prevent droplet infection. In addition to wearing a medical face mask, washing hands frequently and inhibiting one's hands from contacting his eyes, mouth and nose may also prevent from being infected. However, only cleaning the environment thoroughly can effectively avoid most contagious infections.

At present, the infected environment is disinfected by cleaning personnel wearing protective equipment. However, the cleaning personnel risk being infected by viruses in manual disinfection. Further, periodically disinfecting crowded public places would consume much manpower. Besides, humans cannot manually determine which area is seriously infected and needs deep disinfecting, nor can examine whether disinfection has achieved the desired effect one area by one area.

Accordingly, the present invention proposes a cloud-managed autonomous medical vehicle for disinfection to overcome the conventional problems and meet the future requirements. The principles and embodiments thereof will be described in detail below.

### SUMMARY OF THE INVENTION

The primary objective of the present invention is to provide a cloud-managed autonomous medical vehicle for disinfection, which determines the spraying mode according to the detected concentrations of the organic and inorganic materials in the ambient environment, whereby to realize precise cleaning and disinfecting actions of the ambient environment.

Another objective of the present invention is to provide a cloud-managed autonomous medical vehicle for disinfection, wherein movement operating elements are used to enable omnidirectional control and in-place rotation of the mobile carrier, whereby the disinfectant liquid can be sprayed panoramically without rotating a plurality of nozzles, and whereby is avoided the rotation of the nozzles and the tangling of the tubes of the disinfectant liquids.

Yet another objective of the present invention is to provide a cloud- managed autonomous medical vehicle for disinfection, wherein the detector detects the concentrations of the organic and inorganic materials and transmits the detected concentrations to a cloud server, and the cloud server performs data analysis to statistically analyze the concentrations of the organic and inorganic materials in the local sites of the environment.

In order to achieve the abovementioned objectives, the present invention provides a cloud-managed autonomous medical vehicle for disinfection, which comprises a mobile carrier, wherein a disinfectant liquid container is disposed on the mobile carrier and filled with disinfectant liquid; a plurality of nozzles, arranged on the mobile carrier and spraying the disinfectant liquid, wherein the plurality of nozzles are respectively connected with motors, and the motors switch spraying modes of the plurality of nozzles; a detector, scanning an ambient environment, detecting a concentration of an organic material or an inorganic material, sending out a spray start instruction while detecting the concentration is higher than a preset value, sending out a spray stop instruction while detecting the concentration is lower than the preset value, and transmitting the concentration to a cloud server; and a nozzle controller, connected with the detector and the motor, wherein while receiving the spray start instruction, the nozzle controller controls the motors to switch spraying modes of the plurality of nozzles to perform spraying actions according to the spray start instruction; while receiving the spray stop instruction, the nozzle controller turns off the motors to stop the spraying actions.

In one embodiment, the present invention further comprises a navigation device, which is arranged on the mobile carrier, performs cartographical scanning, and drives the mobile carrier to move along a planned path.

In one embodiment, the navigation device includes a Lidar for performing cartographic scanning.

In one embodiment, the navigation device includes a navigation controller, which generates a planned path according to a scanning result of the Lidar.

In one embodiment, the navigation device includes a navigation controller, which receives the planned path from the cloud server, determines a position of the mobile carrier according to the scanning result of the Lidar, and drives the mobile carrier to move along the planned path.

In one embodiment, the planned path includes a plurality of fixed sprayed points; the navigation controller is connected with the detector; while the mobile carrier reaches each of the plurality of fixed sprayed point, the navigation controller stops driving the mobile carrier and informs the detector to scan the ambient environment to enable the plurality of nozzles to perform spraying action.

In one embodiment, the present invention further comprises a retractable bracket, wherein a bottom of the retractable bracket is vertically installed on the mobile carrier; the plurality of nozzles are installed on a top of the retractable bracket.

In one embodiment, the mobile driver or the retractable bracket further includes an elevator controller and an elevator driver; the elevator controller is connected with the detector and the elevator driver; the elevator controller is configured to control the elevator driver according to an altitude-adjusting instruction to adjust a length of the retractable bracket, wherein the spray start instruction includes the altitude-adjusting instruction.

In one embodiment, the spraying modes of the plurality of nozzles comprise a fog-type spraying mode, a droplet-type spraying mode, and a spout-type spraying mode.

In one embodiment, a plurality of movement operating elements is installed on a bottom of the mobile carrier to enable the mobile carrier to move. In one embodiment, the movement operating element is an omnidirectional wheel or a Mecanum-wheel, which enables omnidirectional control and in-place rotation of the mobile carrier.

In one embodiment, the detector transmits the detected concentration to the cloud server in a wireless method.

In one embodiment, the preset value is input into the detector manually or input into the detector from the cloud server from.

### BRIEF DESCRIPTION OF THE DRAWINGS

FIG. 1 is a perspective view schematically showing a cloud-managed autonomous medical vehicle for disinfection according to one embodiment of the present invention.

FIG. 2 is a locally-enlarged view schematically showing nozzles of a cloud-managed autonomous medical vehicle for disinfection according to one embodiment of the present invention.

FIG. 3 is a block diagram showing an architecture of a cloud-managed autonomous medical vehicle for disinfection according to one embodiment of the present invention.

### DETAILED DESCRIPTION OF THE INVENTION

The technical schemes of the embodiments of the present invention will be described clearly and fully in cooperation with the attached drawings. Obviously, the embodiments described in the specification are not all the embodiments of the present invention but only a portion of the embodiments of the present invention.

It should be understood: the terms “comprise” and “include” used in the specification and claims only indicate the existence of characteristics, entireties, steps, operations, elements and/or components but do not exclude the existence or addition of one or more other characteristics, entireties, steps, operations, elements and/or components.

It should be also understood: the terms used in the specification of the present invention are only to describe specified embodiments but not to limit the scope of the present invention. While used in the specification and claims of the present invention, the singular noun, which is described by “one”, “one piece of” or “the”, implies the plural form thereof unless the context indicates another condition clearly.

It should be further understood: the term “and/or” used in the specification and claims of the present invention refers to one or several of the listed items or any possible combination of the listed items, and the present invention includes these combinations.

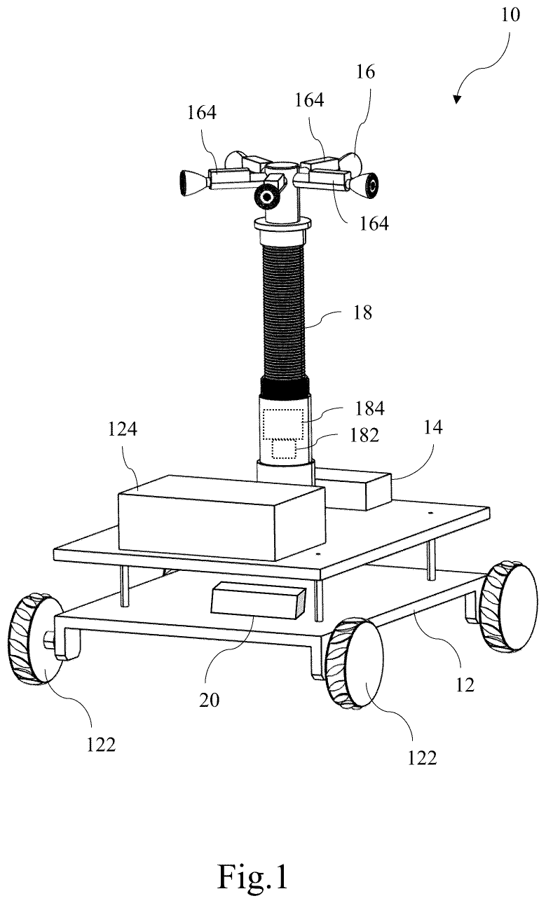

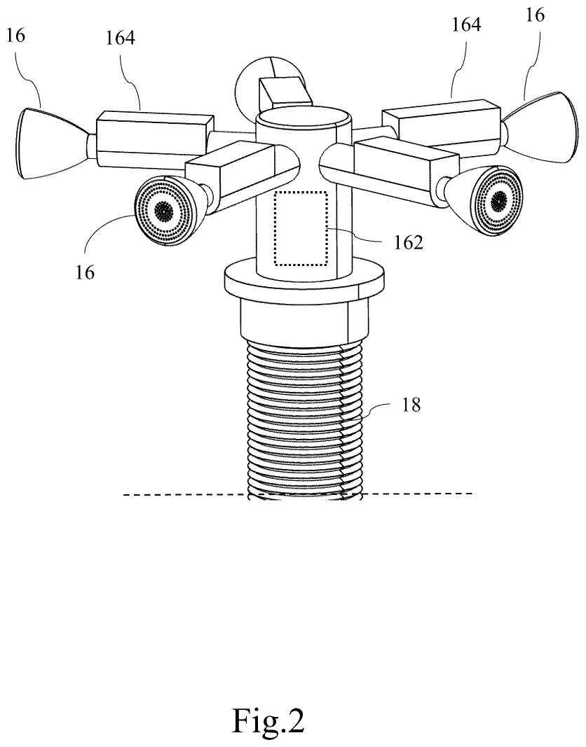

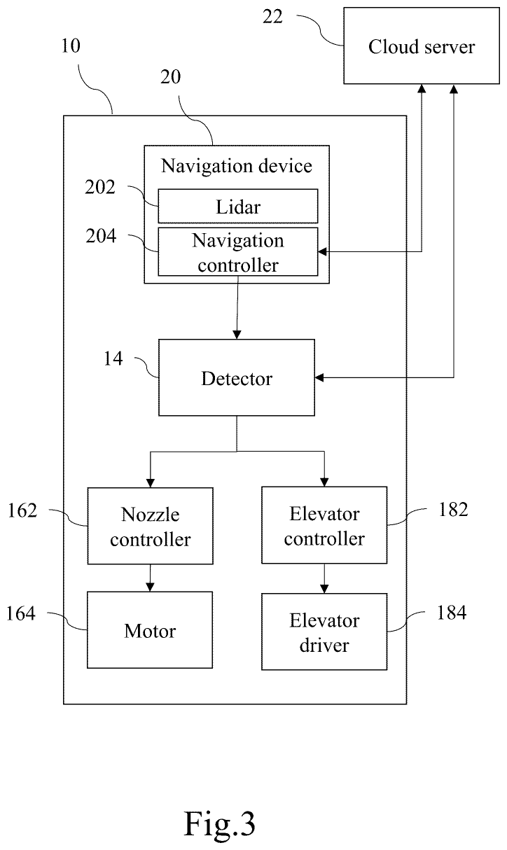

The present invention provides a cloud-managed autonomous medical vehicle for disinfection. FIG. 1 is a perspective view schematically showing a cloud-managed autonomous medical vehicle for disinfection according to one embodiment of the present invention. FIG. 2 is a locally-enlarged view schematically showing nozzles of a cloud-managed autonomous medical vehicle for disinfection according to one embodiment of the present invention. FIG. 3 is a block diagram showing an architecture of a cloud-managed autonomous medical vehicle for disinfection according to one embodiment of the present invention. The cloud-managed autonomous medical vehicle 10 for medical cleaning and disinfecting application of the present invention comprises a mobile carrier 12, a detector 14, a plurality of nozzles 16, and a retractable bracket 18. The detector 14 and the retractable bracket 18 are installed on the mobile carrier 12. The nozzles 16 are installed on the top of the retractable bracket 18. A disinfectant liquid container 124 is also disposed on the mobile carrier 12, containing disinfectant liquid, such as alcohol, diluted bleach, or any one of other disinfecting liquids. The disinfectant liquid container 124 has a pipe (not shown in the drawings), which is connected with the nozzles 16 and transfers the disinfectant liquid to the nozzles 16.

The nozzle 16 is used to spray the disinfectant liquid. In the present invention, the nozzle 16 has several spraying modes, including a fog-type spraying mode, a droplet-type spraying mode, and a spout-type spraying mode. The nozzles 16 are respectively connected with motors 164. Each motor 164 is connected with a nozzle controller 162. The nozzle controller 162 controls the motor 164 to switch the nozzle 16 to a different spraying mode.

The bottom of the retractable bracket 18 is vertically installed on the mobile carrier 12. An elevator controller 182 and an elevator driver 184 are used to control the length of the retractable bracket 18, i.e. to adjust the altitude of the nozzles 16. The elevator driver 184 may be a motor or a hydraulic driver.

The detector 14 is connected with the nozzle controller 162, scanning the ambient environment and detecting a concentration of an organic or inorganic material. A group of control demands are established in the detector 14 beforehand, including a preset value of a concentration lower limit. The control demands may be input into the detector 14 manually or input into the detector 14 by a cloud server 22 from a far end. While the detector 14 detects that the concentration is greater than the preset value, it indicates that the pollution of the organic or inorganic material in the environment has reached such an extent that a cleaning/disinfecting action is required. In such a situation, the detector 14 sends a spraying start instruction, including the spraying mode, to the nozzle controller 162. While receiving the spraying start instruction, the nozzle controller 162 controls the motor 164 to switch the spraying mode and make the nozzle perform a spraying action. Oppositely, while the detector 14 detects that the concentration is lower than or equal to the preset value, it indicates that the pollution of the organic or inorganic material in the environment has been lowered to a safe range. In such a situation, the detector 14 sends a spraying stop instruction to the nozzle controller 162. While receiving the spraying stop instruction, the nozzle controller 162 turns off the motor 164 to make the nozzle 16 stop spraying. The detector 14 further transmits the concentrations to the cloud server 22, and the cloud server 22 performs data analysis to statistically analyze the concentrations of the organic and inorganic materials in the local sites of the environment. In one embodiment, the detector 14 further transmits the concentrations to the cloud server 22 in a wireless method.

The detector 14 is also connected with the elevator controller 182. The elevator controller 182 is connected with the elevator driver 184. While the detector 14 detects that the concentration is greater than the preset value, the detector 14 also detects the spraying range of the nozzle 16. If it is required to raise the nozzle 16 to make the area, which needs cleaning and disinfecting, within the spraying range, the detector 14 sends an altitude-adjusting instruction to the elevation controller 182. The elevation controller 182 controls the elevation driver 184 according to the altitude-adjusting instruction. The elevation driver 184 further controls the retractable bracket 18 to rise or descend.

The mobile carrier 12 has a navigation device 20, which includes a Lidar 202 and a navigation controller 204. The Lidar 202 is connected with the navigation controller 204, performing cartographical scanning of the ambient environment and transmitting the scanned images to the navigation controller 204. The navigation controller 204 plans the path according to the scanning result of the Lidar 202. Besides, the planned path may be established in the cloud server 22 and transmitted to the navigation controller 204; then, the planned path is stored in and executed by the navigation controller 202. The navigation controller 204 may determine the position of the mobile carrier 12 according to the scanning result of the Lidar 202 and compare the position with the planned path. Then, the navigation controller 204 drives the mobile carrier 12 to advance along the planned path.

The navigation controller 204 is connected with the detector 14. In one embodiment, the planned path includes a plurality of fixed sprayed points. While the mobile carrier 12 reaches the fixed sprayed point, the navigation controller 204 stops driving the mobile carrier 12 and informs the detector 14 to scan the ambient environment, and the nozzles 16 perform spraying at the fixed point. While the detector 14 detects that the concentration is lower than the preset value, the mobile carrier 12 continues to move forward.

A plurality of movement operating elements 122 is installed on the bottom of the mobile carrier 12. In one embodiment, the movement operating element 122 is an omnidirectional wheel or a Mecanum-wheel, which enables the mobile carrier 12 to have an omnidirectional control function and an in-place rotation function. Thus, the cloud-managed autonomous medical vehicle 10 for medical cleaning and disinfecting application of the present invention can rotate in place and panoramically spray the disinfectant liquid 360 degrees. In the present invention, the nozzles on the top of the retractable bracket 18 does not rotate lest the tube of the disinfectant liquid tangle or drop.

In a practical operation of the present invention, the cloud-managed autonomous medical vehicle 10 for medical cleaning and disinfecting application is started firstly. Next, the control demands are input into the detector 14, such as the preset values of the detected concentrations of the organic/and inorganic materials. Next, the navigation device 20 performs cartographical scanning and determines the advancing route according to the planned path. While the mobile carrier 12 reaches the fixed sprayed point, the navigation controller 204 stops driving the mobile carrier 12 and informs the detector 14 to scan the ambient environment to detect the concentration of the organic or inorganic material. While the detector 14 detects that the concentration is greater than the preset value, the detector 14 sends a spray start instruction to the nozzle controller 162. According to the spray start instruction, the nozzle controller 162 controls the motor 164 to switch the spraying mode of the nozzles 16 and controls the nozzles to spray. The detector 14 further detects whether the sprayed range completely covers the area where the concentration exceeds the preset value. If the sprayed range does not completely cover the area where the concentration exceeds the preset value, the detector 14 sends out an elevation instruction, and the elevation controller 182 activates the elevation driver 184 according to the elevation instruction to make the retractable bracket 18 rise or descend. While the detector 14 detects that the concentration is lower than or equal to the preset value, the detector 14 sends a spray stop instruction to the nozzle controller 162. According to the spray stop instruction, the nozzle controller 162 turns off the motor 164 to stop spray. After the cleaning/disinfecting operation has been completed at a fixed sprayed point, the navigation device 20 starts the mobile carrier 12 to move to the next fixed sprayed point where the nozzles 16 will perform spraying. Besides, the detector 14 transmits the concentrations to the cloud server 22.

The embodiments described above are only to exemplify the present invention but not to limit the scope of the present invention. The embodiments involving equivalent modification or variation made according to the characteristics and spirit of the specification or claims are to be also included by the scope of the present invention.

What is claimed is:

1. A cloud-managed autonomous medical vehicle for disinfection comprising:
a mobile carrier, wherein a disinfectant liquid container is disposed on the mobile carrier and filled with disinfectant liquid;
a plurality of nozzles arranged on the mobile carrier and used to spraying the disinfectant liquid, wherein the plurality of nozzles are respectively connected with motors to switch spraying modes of the plurality of nozzles;
a detector scanning an ambient environment to detect a concentration of an organic material or an inorganic material, sending out a spray start instruction while detecting the concentration is higher than a preset value or a spray stop instruction while detecting the concentration is lower than or equal to the preset value, and transmitting the concentration to a cloud server; and
a nozzle controller connected with the detector and the motors, wherein:
while receiving the spray start instruction, the nozzle controller controls the motors to switch spraying modes of the plurality of nozzles to perform spraying action according to the spray start instruction;
while receiving the spray stop instruction, the nozzle controller turns off the motors to stop the spraying action.

2. The cloud-managed autonomous medical vehicle for disinfection according to claim 1, further comprising a navigation device arranged on the mobile carrier, the navigation device performing cartographic scanning and drives the mobile carrier to move along a planned path.

3. The cloud-managed autonomous medical vehicle for disinfection according to claim 2, wherein the navigation device comprises a Lidar for performing cartographic scanning.

4. The cloud-managed autonomous medical vehicle for disinfection according to claim 3, wherein the navigation device comprises a navigation controller configured to generate the planned path according to a scanning result of the Lidar.

5. The cloud-managed autonomous medical vehicle for disinfection according to claim 3, wherein the navigation device comprises a navigation controller connected with the cloud server, wherein the navigation controller is configured to receive the planned path from the cloud server, determine a position of the mobile carrier according to a scanning result of the Lidar, and drive the mobile carrier to move along the planned path.

6. The cloud-managed autonomous medical vehicle for disinfection according to claim 4, wherein the planned path comprises a plurality of fixed sprayed points, and the navigation controller is connected with the detector; while the mobile carrier reaches each of the plurality of fixed sprayed points, the navigation controller stops driving the mobile carrier and informs the detector to scan the ambient environment, enabling the plurality of nozzles to perform spraying action.

7. The cloud-managed autonomous medical vehicle for disinfection according to claim 5, wherein the planned path comprises a plurality of fixed sprayed points, and the navigation controller is connected with the detector; while the mobile carrier reaches each of the plurality of fixed sprayed points, the navigation controller stops driving the mobile carrier and informs the detector to scan the ambient environment, enabling the plurality of nozzles to perform spraying action.

8. The cloud-managed autonomous medical vehicle for disinfection according to claim 1, further comprising a retractable bracket of which a bottom is vertically installed on the mobile carrier and a top is installed with the plurality of nozzles.

9. The cloud-managed autonomous medical vehicle for disinfection according to claim 8, wherein the mobile driver or the retractable bracket further includes an elevator controller and an elevator driver; the elevator controller is connected with the detector and the elevator driver, and the elevator controller is configured to control the elevator driver according to an altitude-adjusting instruction to adjust a length of the retractable bracket; the spray start instruction includes the altitude-adjusting instruction.

10. The cloud-managed autonomous medical vehicle for disinfection according to claim 1, wherein the spraying modes of the plurality of nozzles comprise a fog-type spraying mode, a droplet-type spraying mode, and a spout-type spraying mode.

11. The cloud-managed autonomous medical vehicle for disinfection according to claim 1, wherein a plurality of movement operating elements is installed on a bottom of the mobile carrier to enable the mobile carrier to move.

12. The cloud-managed autonomous medical vehicle for disinfection according to claim 11, wherein the movement operating element is an omnidirectional wheel or a Mecanum-wheel, enabling omnidirectional control and in-place rotation of the mobile carrier.

13. The cloud-managed autonomous medical vehicle for disinfection according to claim 1, wherein the detector transmits the concentration to the cloud server in a wireless method.

14. The cloud-managed autonomous medical vehicle for disinfection according to claim 1, wherein the preset value is input into the detector manually or input from the cloud server.
